(19) United States
(12) Patent Application Publication
(10) Pub. No.: US 2024/0099866 A1
(43) Pub. Date: Mar. 28, 2024

(54) Coated Medical Devices

(71) Applicant: phenox GmbH, Bochum (DE)

(72) Inventors: Hans HENKES, Stuttgart (DE); Hermann MONSTADT, Bochum (DE); Ralf HANNES, Dortmund (DE)

(73) Assignee: phenox GmbH, Bochum (DE)

(21) Appl. No.: 18/274,344

(22) PCT Filed: Jan. 18, 2022
(86) PCT No.: PCT/EP2022/050972

§ 371 Date: Jul. 26, 2023

(30) Foreign Application Priority Data
Jan. 26, 2021 (DE) DE102021101691.6
Feb. 3, 2021 (DE) DE102021102458.7

(51) Int. Cl. A61F 2/90; A61L 31/08; A61L 31/10

(52) U.S. Cl. CPC A61F 2/90; A61L 31/082; A61L 31/10; A61F 2210/0076; A61F 2220/0025; A61F 2230/0063; A61F 2250/0017; A61F 2310/00395; A61L 2420/08

(57) ABSTRACT

A device (1) with a stent structure (2) wherein the stent structure (2), preferably at its proximal end, is connected to an insertion aid (3), and wherein the device (1) is deployable for the treatment of a vasospasm and the stent structure (2) is designed so as to be detachable from the insertion aid (3), with at least portions of the stent structure (2) being provided with a coating and this coating comprising a functional layer, with said functional layer containing at least one sugar alcohol and/or being formed by an oligo- or polymerization of monosaccharides functionalized with polymerizable groups. Furthermore, the invention also relates to a relevant method for the treatment of vasospasms.

The invention relates to a device having a stent structure intended for the insertion into blood vessels of the human or animal body, wherein the stent structure assumes an expanded state during which it is in contact with the inner wall of the blood vessel and a diameter-reduced state during which the stent structure being located in a microcatheter can be moved through the blood vessel, wherein the stent structure preferably being connected at its proximal end to an insertion aid.

Vascular endoprostheses, or stents, are frequently employed for the treatment of vasoconstrictions and are permanently implanted at the site of the vascular stenosis to keep the vessel open. Typically, stents have a tubular structure and are either made by laser cutting to achieve a surface consisting of struts with openings between them or they consist of a wire braiding. Stents can be delivered to the target site through a catheter and expanded there; in the case of self-expanding stents made of shape memory materials, this expansion and contact with the inner vessel wall occurs autonomously. Alternatively, stents may also be caused to expand with the aid of balloons onto which the stent is crimp-mounted, or by other mechanical methods. After final placement, only the stent remains at the target site; catheter, guide or pusher wires, and other auxiliary means are removed from the blood vessel system.

Implants of basically similar design are also used for the occlusion of aneurysms in that they are placed in front of the neck of an aneurysm. However, such flow diverters have a higher surface density than stents for the elimination of stenoses. An example of a flow diverter has been described in publication WO 2008/107172 A1.

Vasospasm is a spasmodic constriction of a blood vessel. Vasospasms involve the risk of blood no longer being supplied in sufficient quantities to downstream vessels (ischemia) which may lead to necrosis of the tissue thus cut off from perfusion. Especially in the cerebral area, vasospasm can occur a few days after subarachnoid hemorrhage (SAH), quite frequently as a result of the rupture of an aneurysm. Other causes of subarachnoid hemorrhage are craniocerebral traumata and bleeding from vascular malformations or tumors. Blood that has ingressed into the subarachnoid space washes around the vessels located there and is regarded as the most important triggering factor of vasospasm. Approximately 60% of all SAH patients experience vasospasm to a greater or lesser degree between the fifth and twentieth day after hemorrhage. If arterial vessels are severely constricted, the dependent brain tissue becomes undersupplied and may suffer irreversible damage (cerebral infarction). Approximately 15 to 20% of all patients who primarily survived SAH experience permanent neurologic damage with resulting disability. Approximately 5% of the primarily surviving SAH patients subsequently die as a result of cerebral vasospasm. In this respect, vasospasm is one of the main reasons for apoplexy and even deaths occurring after rupture of an aneurysm and/or bleeding from the same or surgery in this area.

Usually, vasospasm is treated with drugs, especially calcium channel blockers or medication that increases the level of NO in the blood. An example of a calcium channel blocker is nimodipine which is frequently used after subarachnoid hemorrhages with a view to preventing vasospasms. However, such a medication-based treatment is associated with not inconsiderable side effects and, moreover, is costly and time-consuming.

Other options for the treatment of vasospasm include intensive care measures such as raising the arterial blood pressure and increasing the circulating blood volume, dilating constricted vessels with the help of a balloon, blocking the stellate ganglion, and surgically eliminate sympathetic nerve fibers (sympathicolysis). These treatment methods are individually inconsistent in their effectiveness, in some cases very complex, and often do not have a sufficiently long-lasting effect. Although blockade of the stellate ganglion and surgical sympathicolysis are effective because sympathetic nerve fibers in the wall of the cerebral arteries are significantly involved in the development of cerebral vasospasm, these procedures are, however, inadequate for the full prevention and treatment of cerebral vasospasm because the blockade of the stellate ganglion lasts only a few hours and surgical sympathectomy is limited to a narrowly circumscribed vascular segment that must be surgically prepared for this purpose.

From WO 2017/207689 A1 a device for the treatment of a vasospasm is known, which essentially involves a stent structure, which, however, does not remain permanently in the vascular system, but is navigated to the location of the vasospasm and expanded there, to be subsequently retracted again. Such treatment must often be repeated at intervals of a few days or weeks.

However, it would be desirable to have a device available for the treatment of vasospasm that can remain permanently in the body as an implant to treat the vasospasm and, in particular, to prevent the recurrence of vasospasm.

Previously, it was thought that a permanent implant was inappropriate for the treatment of vasospasm because the insertion of an implant carries the risk of platelet aggregation. The adhesion of platelets as well as platelet aggregation and thus the formation of blood clots, so-called thrombi, can be observed with permanent implants because the platelets adhere to the surface of the inserted implant marked by the body's own proteins (platelet adhesion), which can subsequently lead to the formation of a thrombus (platelet aggregation). In most cases, this is treated with platelet aggregation inhibitors such as acetylsalicylic acid (ASA), clopidogrel, prasugel or ticagrelor.

It is thus the objective of the invention to provide a device that is suitable for the treatment of vasospasm and can remain permanently as an implant in the body, preferably without needing additional administration of platelet aggregation inhibitors.

As proposed by the invention, this objective is achieved by a device with a stent structure which is intended for insertion into blood vessels of the human or animal body, wherein the stent structure has an expanded state in which it is in contact with the inner wall of the blood vessel and a diameter-reduced state in which it is movable through the blood vessel within a microcatheter, wherein the stent structure, preferably at its proximal end, is connected to an insertion aid, and wherein the device is deployable for the treatment of a vasospasm and the stent structure is designed so as to be detachable from the insertion aid, wherein at least portions of the stent structure are provided with a coating and this coating comprises a functional layer, with said functional layer containing at least one sugar alcohol and/or is formed by an oligo- or polymerization of monosaccharides functionalized with polymerizable groups.

The inventive device essentially comprises at least one substrate serving as base of the actual device as well as a functional layer. The functional layer imparts the desired properties to the device and has a biomimetic or biorepulsive effect.

Preferably, the functional layer substantially comprises a complex, highly branched, hydrophilic matrix with a plurality of molecules each having a main chain as polymer backbone and each having a plurality of side chains. The main and/or side chains may form bonds with other main and/or side chains. Other matrix-forming mono-, oligo- and polymers may be incorporated into these main and side chains without themselves being covalently bonded to the substrate. The saccharides forming the functional layer, with sugar alcohols also being regarded as saccharides within the meaning of the invention, are functionalized with polymerizable groups that are capable of bonding to the surface of the stent structure and bring about polymerization.

The polymerizable groups via which the saccharides are functionalized may have reactive multiple bonds, especially reactive double bonds. Polymerization can thus take place via the double bonds. In particular, it may be an acrylic or methacrylic group whose suitability for polymerization reactions is known to persons skilled in the art. Other groups suitable for polymerization, such as vinyl or allyl, can also be used. The main chain thus exhibits at least partially polymerized vinyl, allyl, acrylic or methacrylic compounds or derivatives thereof and/or isomers thereof or combinations thereof. Thus, oligo- or polymerization of the saccharides normally takes place via the polymerizable groups through which the saccharides are functionalized; on the other hand, a new formation of glycosidic bonds does not take place usually.

The side chains particularly comprise mono- and/or oligosaccharides, with reduction products of mono- or oligosaccharides being also understood as such, in particular sugar alcohols (alditols). Aside from this and for the purposes of the present invention, oxidized mono- and/or oligosaccharides may also occur, with the oxidized form also being understood as mono- or oligosaccharide.

Without wishing to be bound by any particular theory, the advantage of the coating proposed by the invention is seen in the fact that the functional layer has biomimetic, respectively biorepulsive, properties and is not recognized by platelets as foreign to the body, but rather as endogenous. Accordingly, the functional layer proposed by the invention does not trigger any reaction of the thrombocytes, especially does not give rise to adhesion or aggregation reactions.

The biomimetic effect of the inventive coating is attributed to the fact that the functional layer that is claimed by the invention imitates human glycocalyx. Glycocalyx covers the cells of blood vessels with a kind of mucus layer and consists of various polysaccharides that are covalently linked to the membrane proteins and membrane lipids. Accordingly, glycoproteins and glycolipids are obtained.

It is advantageous for the high biomimetic effect of the coating as proposed by the invention—and in particular of the functional layer—if the polymerization of the reactants of the functional layer solution essentially occurs only after the functional layer solution has been applied to the substrate. As a result, polymerization of the reactants produces a complex layer that is so similar to the glycocalyx that the adhesion of platelets to surfaces provided with the inventive coating is significantly lower than to uncoated surfaces.

The biorepulsive effect of the coating according to the invention is based on the principle of steric repulsion. Presumably, the space available to the oligomers and polymers on the surface is reduced when a protein intrudes on this space, i.e., an approaching protein forces the oligomers and polymers on the surface to adopt a more energetically unfavorable conformation. This results in an overall repulsive force acting against proteins. It is also conceivable that the displacement of water molecules from the coating results in a repulsive osmotic force acting against proteins.

As regards thrombocyte/platelet adhesion, this principle of action means that the adherence of thrombocytes is prevented because there are no or only a few proteins on the surface that are suitable for bonding so that platelet adhesion is significantly reduced.

The stent structure, which is cylindrical in at least some areas and preferably as a whole, generally has openings distributed over the circumferential surface of the cylinder. In other words, a lattice or mesh structure is created, built up of struts, so that a plurality of openings or meshes are formed on the circumferential surface of the basic cylindrical structure.

A stent structure composed of interconnected webs or struts can be produced by laser cutting in a basically known manner; in this context, one also speaks of cut structures. In this way, a plurality of openings or a mesh structure is created within the stent structure, with the openings being distributed over the circumference of the stent structure. Other manufacturing processes may be adopted as well, such as galvanic or lithographic production, 3D printing or rapid prototyping.

Alternatively, the stent structure may also be a mesh-like structure comprising wires in the form of a braiding. The wires in this case typically extend helically along the longitudinal axis, with intersecting opposed wires extending above and below each other at points of intersection resulting in honeycomb-like openings being created between the wires. The total number of wires preferably ranges between 8 and 64. As wires forming the mesh structure individual wires made of metal may be employed but it is also possible to provide strands, i.e., several wires of small diameter arranged so as to form a filament, preferably twisted around each other.

The term “aperture” or opening refers to the lattice structure, regardless of whether the aperture is isolated from the environment by a membrane, i.e., even an aperture covered by a membrane is called an aperture or opening. As desired, a membrane can be applied to the outside or inside of the lattice structure. It is also possible to embed the lattice structure in a membrane. The membranes can be made of a polymer material such as polytetrafluoroethylene, polyester, polyamides, polyurethanes, polyolefins or polysulfones. Especially preferred are polycarbonate urethanes (PCU).

An advantage of a stent structure comprising interconnected webs or struts that in particular are produced by laser cutting techniques over a mesh structure comprising wires is that during the expansion process a stent structure of struts will be less prone to longitudinal contraction than a mesh structure. Longitudinal contraction should be kept to a minimum because the stent structure exerts additional stress on the surrounding vessel wall during longitudinal contraction. Due to the fact that vasospasm is especially caused by stimuli acting on the vessel any additional stress has to be avoided in the treatment of vasospasm.

A stent structure of interconnected struts moreover offers advantages in that the radial force exerted by such a stent structure of otherwise comparable construction, struts/wire density and struts/wire thickness is higher than that of a mesh structure comprising wires. The reason for this is that the struts are permanently attached at the points of intersection, whereas the wires of a mesh structure usually only extend over and under each other.

The struts or wires may have a round, oval, square, rectangular or trapezoidal cross section, with edges of square, rectangular or trapezoidal cross sections being advantageously rounded off. In addition, it is thought expedient to process the stent structure by electropolishing to make it smoother and rounder and thus render it less traumatic. In addition, the risk of adhesion of germs or other contaminants is reduced. Flat struts/wires in the form of thin strips, especially metal strips may be employed as well.

The openings thus formed within the stent structure between the individual struts or wires should have an inscribed circle diameter ranging between 0.1 and 6 mm, where under inscribed circle diameter the diameter of the largest possible circle is to be understood that can be placed in the opening. The data refer to the stent structure in the expanded state, i.e., the state that the stent structure assumes when it is not subject to any external constraints or limitations. Depending on the diameter of the blood vessels in which the implant is implanted, the expanded state in the vasculature may differ from the expanded state existing in the absence of external constraints because the implant may not be able to assume its fully expanded state.

Preferred are openings having an inscribed circle diameter of 1 mm, which translates into a relatively coarse-meshed stent structure, because such a structure is capable of exerting a radial force of a magnitude suitable for the treatment of vasospasm.

The openings created in the stent structure may be closed all around, that is, surrounded by struts or wires without interruptions (so-called “closed cell design”). However, an “open-cell design” is preferred, in which at least some struts/wires have an interruption so that the cells formed by the struts/wires are at least partially open, i.e., not completely closed. Such an open-cell design exhibits greater flexibility, which can be advantageous in highly tortuous blood vessels. Furthermore, stent structures comprising a closed-cell design have a tendency to adopt a straight-line elongated configuration, which may place some stress on the blood vessel, especially if the vessel itself has a more curvilinear course.

Moreover, in the interest of producing a radial force of suitable magnitude it is considered expedient to use struts or wires having a relatively large cross section or diameter, i.e., using relatively massive struts/wires. For example, when using struts or wires with a substantially rectangular cross section, a height and a width of the struts/wires of between 30 and 300 μm have proven to be advantageous, with a rectangular cross section with rounded edges also being considered to be substantially rectangular. In the event of a round cross section the diameter should range between 30 and 300 μm.

However, as already mentioned, the stent structure can also be a mesh structure of wires forming a braid, and in this context, one also speaks of a braided stent structure.

Loose wire ends may be present at the proximal and distal ends of the stent structure, respectively, but these should preferably be atraumatic to avoid injury to the blood vessel. The atraumatic design of the wire ends can be achieved, for example, by rounding the wire ends off. Another option is to allow the wires to form a loop at one or both ends of the stent structure and then guide them back into the braid. Accordingly, the end of the stent structure no longer has free wire ends, reducing the risk of injury to the blood vessel wall.

The density of the struts or wires of the stent structure can be such that the stent structure resembles a conventional stent as it is used to keep vessels open, but it may as well be significantly higher, in which case the stent structure more closely resembles a flow diverter of the type placed in front of aneurysms to cut off the aneurysm from the flow of blood. Flow diverters have a higher surface coverage, which in the expanded state often ranges between 20 and 65%, i.e., a correspondingly high proportion of the total surface area of the flow diverter is made of material in the form of struts/wires between which the openings are located. The above comments on the design of the stent structure, struts and wires, and the course or configuration of the same, etc., apply regardless of whether the stent structure is more like a conventional stent or a flow diverter.

Even though the aforementioned stent structure provided with the biomimetic coating described hereinbefore is intended to remain in the blood vessel permanently, a relevant stent structure can also be employed for only a short-term insertion into and expansion within the blood vessel. For a stent structure that is not intended to remain permanently in the blood vessel but is removed after a few minutes at a time, a detachment point between the stent structure and the insertion aid is not absolutely required. Such an embodiment of the device with a stent structure at least partially provided with the described coating is also understood to be claimed in accordance with the invention. Nevertheless, a detachment point may be beneficial to provide the treating physician with different options as the relevant situation may require, i.e., to retract the stent structure or, if retraction causes problems or the physician decides for other reasons that the stent structure should remain in the blood vessel permanently, to disconnect the stent structure at the detachment point.

Typically, the insertion aid is provided in the form of an insertion or pusher wire as it is known for use with implants. In the case of implants that are intended to remain permanently in the vascular system, the insertion aid is attached to the implant via a detachment point, and said detachment point may be designed to permit mechanical, thermal, or electrolytic disconnection. The device proposed by the invention has at least one such detachment point, with a single detachment point being preferred to simplify disconnection/release. The insertion aid is preferably made of stainless steel, nitinol or a cobalt-chromium alloy.

The insertion aid or insertion wire is preferably attached at the proximal end of the stent structure radially outward. In other words, the connection between insertion aid and stent structure is not in the center of the stent structure but arranged eccentrically at or near the inner wall of the vessel. In this way, the flow of blood is impeded as little as possible. Moreover, an eccentric disposition of the insertion aid facilitates retraction of the device into the microcatheter should this prove necessary.

The detachment point or points are preferably designed to be electrolytically corrodible. In this case, the at least partial dissolution of the detachment point is achieved by applying an electrical voltage to the detachment point making use of a suitable voltage/power source. The electrolytic dissolution method causes the detachment point to be electrolytically corroded by applying a voltage resulting in the implant to disconnect from the insertion aid. Usually, direct current is used, with a low amperage (<3 mA) being sufficient. The detachment point is usually made of metal and, when an electrical voltage is applied, acts as anode where oxidation and thus dissolution of the metal takes place.

To avoid anodic oxidation of the implant, it should be electrically isolated from the detachment point and the insertion aid. The electrolytic detachment of implants is well known practice in the state of the art, for example for occlusion coils used for the purpose of closing off aneurysms, cf. WO 2011/147567 A1, for example. The principle is based on the fact that when a voltage is applied, an appropriately designed detachment point made of suitable material, in particular metal, is dissolved as a rule by anodic oxidation at least to such an extent that the areas of the implant located distally to the relevant detachment point are released. The detachment point can be made, for example, of stainless steel, magnesium, magnesium alloys or a cobalt-chromium alloy. A particularly preferred magnesium alloy is Resoloy®, developed by the company MeKo from Sarstedt/Germany (cf. WO 2013/024125 A1). This is an alloy consisting of magnesium and, inter alia, of lanthanides, in particular dysprosium. Another advantage of using magnesium and magnesium alloys is that magnesium residues remaining in the body are physiologically unproblematic.

While the detachment point serves as anode, the cathode can, for example, be positioned on the body surface. Alternatively, another area of the device may also be used as cathode. The detachment point must, of course, be connected in an electrically conductive manner with the power source. The insertion aid, in particular the insertion wire itself, can serve as conductor. Since, with the cathode being placed on the body surface, the corrosion current that sets in is controlled by the area of the cathode, the area of the cathode should be chosen to be significantly larger than the area of the anode. To some extent, the dissolution rate at which the detachment point is dissolved can be controlled by appropriately sizing the cathode surface relative to the anode surface. The device proposed by the invention may thus also comprise a voltage source and, where applicable or appropriate, an electrode that can be placed on the surface of the body.

As an alternative to a detachment point which is to be dissolved electrolytically, other detachment points known from prior art may also be employed, in particular detachment points that can be separated mechanically, thermally or chemically. In the event of a mechanical detachment/severance, a form-closed, force-closed or friction fit typically exists, that is broken when the stent structure is released causing the stent structure to separate from the insertion aid. In the event of a thermal detachment point, the connection can be broken by heating the detachment point, causing it to soften or melt so that severance is achieved. Another option is to make use of chemical severance in such a way that the detachment is brought about by a chemical reaction occurring at the point of detachment.

Different detachment types, for example electrolytic and mechanical detachment, may also be combined. For this purpose, a mechanical connection, in particular brought about by a form fit, is established between the elements, and this connection remains in place until an element maintaining the mechanical connection is electrolytically corroded.

Preferably, the stent structure is of self-expanding design capable of autonomously transition to the expanded state upon release from the microcatheter. To achieve this, a stent structure is of advantage that is made of a material having shape-memory properties, and in particular the use of nickel-titanium alloys known under the tradename of nitinol has proven its worth. However, also conceivable are polymers having shape-memory characteristics or other alloys.

The device proposed by the invention may, in particular, be used in the neurovascular field; it may, however, also be employed in the cardiovascular or peripheral region.

Generally, the treatment is performed by advancing the device of the invention within a microcatheter to the target site, i.e., the site where vasospasm has occurred. Following this, the microcatheter is retracted in proximal direction causing the release of the stent structure which then expands and touches the inner wall of the vessel, thus treating the vasospasm. At this point, the stent structure is left in place permanently or, if temporarily, for a period of time, typically 1 to 10 minutes. If the stent structure is not to remain permanently in the blood vessel, the microcatheter is then again moved distally to fold in the stent structure, and the microcatheter is withdrawn together with the device. The treatment may be repeated on several days in succession.

The terms “proximal” and “distal” are to be understood such that they refer as proximal to parts that point towards the treating physician when inserting the device, and as distal to parts that point away from the treating physician. Typically, the device is thus moved forward in distal direction with the aid of a microcatheter. The term “axial” refers to the longitudinal axis of the device extending from proximal to distal while the term “radial” denotes levels/planes extending vertically thereto.

Parallel to this treatment carried out with the aid of the inventive device, a drug treatment can also be carried out, for example by administering nimodipine. This may in particular be applied intra-arterially at the location where vasospasm has occurred.

As a rule, the stent structure is designed so as to be open at both ends in order to disturb the flow of blood as little as possible and to prevent an undersupply of subsequent blood vessels and the tissue supplied by them. A stent structure that is not intended to remain permanently in the blood vessel may also be closed at the distal end; a closed structure arranged at the distal end is more atraumatic. In this context, open is understood to mean that there are no struts or wires arranged at the respective end of the stent structure and that struts/wires are limited to the outer circumference of the stent structure. However, in case a closed end is provided, struts or wires also exist in the center of the stent structure. Since openings still exist between the struts or wires even when the distal end is closed, this end is not completely impervious; the blood flow can still pass through the respective openings.

The force the expanding stent structure exerts radially outward on the inner wall of the vessel should range between 2 and 30 N/m, preferably between 5 and 10 N/m based on a stent structure diameter of 2.00 mm. The radial force specified in this case refers to the force radially exerted per unit of length, i.e., is to be viewed as relative radial force. In this case, only that part of the stent structure is to be taken into account that is in contact with the inner wall of the vessel and is thus capable of exerting forces on it (effective length). Along the effective length, the stent structure must cover a minimum of 50% of an envelope arranged around the stent structure. In contrast, the absolute radial force denotes the value applicable to the entire stent structure.

The exerted radial force (chronic outward force, COF) is determined by means of a vee block test as described below:

The setup of the vee block test comprises two polymethyl methacrylate (PMMA) blocks, each provided with a milled and smoothly polished 90° vee-groove. These vee blocks are placed one on top of the other in such a way that when in contact a hollow space of square cross section is created between the blocks. Whereas one of the vee blocks is firmly secured, the other is equipped with a force sensor.

COF describes the force the stent structure exerts on the blood vessel or, in the test, the vee blocks during its self-expansion. For radial force determination, the stent structure located inside a transportation hose or microcatheter is placed centrally between the vee blocks. Following this, the transportation hose/microcatheter is retracted causing the stent structure to be released. Due to its self-expanding properties the structure folds out and the radial force it then produces can be measured by means of the force sensor connected to one of the vee blocks and then further evaluated. To enable stent structures of different lengths to be compared the relative radial force is calculated as follows:

$$COFrel . = \frac{COFabs .}{eff . length of stent structure}$$

According to an advantageous embodiment, the radial force exerted by the stent structure in the expanded state is substantially constant along its length, i.e., in the proximal section and in the distal section, the radial force is equal to that of the middle section. In contrast, in conventional, uniformly constructed stents, the actual radial force exerted in the proximal and distal sections is usually weaker than in the middle section. Therefore, it is expedient to selectively increase the radial force in the proximal and distal sections to create a stent structure in which the radial force in the expanded state is essentially constant over the effective length, with the proximal end of the stent structure, where the struts or wires typically no longer fully abut the inner wall of the vessel, being disregarded in radial force considerations. Therefore, the proximal end thus refers to that portion of the stent structure which is located most proximal and no longer is part of the effective length, and where the struts/wires converge towards the insertion wire. Typically, the length of this proximal end is between 8 and 10 mm, i.e., the total length of the stent structure is longer than the effective length of the stent structure by approximately this amount.

To bring about the increased radial force in the proximal and distal sections, the struts or wires may be designed to have a larger cross section here than in the middle section, with the struts/wires are thus being more massive, which fully or partially compensates for the inherent tendency of a stent structure to exert higher radial forces in the middle section.

Alternatively or additionally, the density of the struts or wires in the proximal section may be provided to be higher than in the middle section. This measure also fully or partially compensates for the proximal or distal decrease in radial force observed with conventional stents.

Another possibility is to provide a slit in the stent structure extending helically over the stent structure's circumferential surface or in longitudinal direction along the circumferential surface of the stent structure. In this case, individual struts or wires may span the slit with a view to influencing the radial force characteristics.

Typically, the diameter of the stent structure in freely expanded state is in the range of between 2 and 8 mm, preferably ranges between 4 and 6 mm. The total length of the stent structure in expanded state usually ranges between 5 and 50 mm, preferably between 10 and 45 mm, further preferred between 20 and 40 mm. The effective length, i.e., the length of the stent structure in expanded state that actually exerts radial forces on the inner wall of the vessel, is in most cases approx. 8 to 10 mm shorter.

In the case of a stent structure made of struts, for example, the structure can be cut from a tube having a wall thickness ranging between 25 and 70 μm; in the case of a mesh structure comprising interwoven wires, the wire thickness is preferably between 20 and 70 μm. For example, a microcatheter by means of which the device in compressed state can be navigated to its target site has an internal diameter of between 0.4 and 0.9 mm.

Another possibility is to integrate electrical conductors into the stent structure by means of which electrical pulses, radio frequency pulses or ultrasound pulses can be applied to nerve fibers extending in the vascular wall of the blood vessel with a view to temporarily or permanently diminishing the function of the nerve fibers and in this way to prevent or treat vasospasm. Such a principle has been described in publication WO 2018/046592 A1 and is based on using a stent structure for the endovascular denervation of brain-supplying arteries.

Physically, pulses can be applied to the nerve fibers in the form of high-frequency (HF) signals, direct current, alternating current or ultrasound. As a rule, denervation is ultimately based on heating of the vessel wall, which leads to elimination or impairment of the function of the nerve fibers. The use of high-frequency or ultrasound pulses is preferred in so far as this allows energy maxima to be generated in the depth of the surrounding vessel wall, so that specifically the nerve fibers are damaged rather than the entire wall of the vessel. The nerve fibers involved here are those of the sympathetic nervous system.

It is expedient to provide the device with one or several radiopaque markers allowing the attending physician to visualize the treatment. The radiopaque markers may, for example, be of platinum, palladium, platinum-iridium, tantalum, gold, tungsten or other metals opaque to radiation. For example, radiopaque coils may be arranged in the device at various points. It is also possible to provide the stent structure, in particular the struts or wires of the stent structure, with a coating comprising a radiopaque material, for example a gold coating. This coating may have a thickness of between 1 and 6 μm, for example. Coating with a radiopaque material need not cover the entire stent structure; it is of particular importance in the areas of the stent structure that contact the inner vessel wall, i.e., essentially in the cylindrical portion of the stent structure. Nevertheless, even when applying a radiopaque coating it is considered expedient to additionally arrange one or several radiopaque markers in the device, in particular at the distal end of the stent structure.

An additional option is to use struts made of a metal having shape memory properties, especially an appropriate nickel-titanium alloy, which at least in part have a platinum core. Such struts are known as DFT (drawn filled tubing) wires. In this way, the advantageous properties of nickel-titanium on the one hand, namely imparting shape memory properties, are combined, on the other hand, with the beneficial properties offered by platinum, namely ensuring X-ray visibility.

In addition to the device according to the invention, the invention also relates to a method for vasospasm treatment, wherein a device of the type described above is used. Said method provides for the stent structure of the device to be navigated to the location of the vasospasm by means of the insertion aid and expanded there, which is usually done by retracting the microcatheter in which the device is housed in a proximal direction. The stent structure is then detached from the insertion aid, which can be done electrolytically, i.e., by applying an electrical voltage to the detachment point arranged between the stent structure and the insertion aid.

Before the stent structure is advanced by a microcatheter to the target position, a relatively large-lumen guide catheter is often used initially, through which in turn the small-lumen microcatheter is advanced further distally. For neurovascular applications, for example, advancement can be made through the guide catheter from the groin to the carotid artery, and further advancement is then made only through the microcatheter.

It is also conceivable to place the stent structure only temporarily at the position of the vasospasm. In this case, the stent structure is left in the expanded state at the position of vasospasm for only a few minutes, preferably for 1 to 10 min. The stent structure is then removed from the blood vessel. For this purpose, the microcatheter can be advanced in the distal direction to again fold up and collapse the stent structure and accommodate it in the microcatheter. Following this, microcatheter and device can be withdrawn and removed from the blood vessel system. It is advisable to repeat the treatment as described several days in succession to continue the vasospasm treatment.

A design of the device as well as a respective coating, as described hereinbefore in connection with the treatment of a vasospasm, can also be used for other purposes, including in particular the treatment of a stenosis (vasoconstriction) or the treatment of aneurysms. In the event the treatment of a stenosis is involved, the device virtually serves as a customary stent, with the stent being provided however with the described coating to prevent the accumulation of platelets and thus the formation of blood clots that would jeopardize the success of treatment. As described above, the stent structure can be (laser) cut, making use of a closed-cell or at least a partial open-cell design. Optionally, a braided stent structure provided with loose wire ends located at the proximal and/or distal end may also be employed, in which case, however, the wires at the proximal and/or distal end of the stent structure must be routed back into the braiding.

Another alternative application is to use it as a flow diverter for the treatment of aneurysms. What has been said above with respect to the basic design and coating still applies, however, the surface coverage resp. surface density of a flow diverter typically exceeds that of a customary stent. The flow diverter is positioned in front of the neck of the aneurysm ensuring the blood flow is kept away from the aneurysm and in this way finally leading to a deterioration/atrophy of the aneurysm.

Another possible function a stent structure or flow diverter placed in front of an aneurysm may serve is to prevent the escape of occlusion agents such as occlusion coils incorporated into an aneurysm. Such an exit or escape of occlusion agents from the aneurysm may lead to undesirable consequences, for example resulting in occlusion agents being carried by the flow of blood to areas located further distally where they may cause the obstruction of a blood vessel or injury to the blood vessel wall. For this purpose, the stent structure can be permanently implanted in the blood vessel, but it is also possible to place a device only temporarily in front of the aneurysm after insertion of a microcatheter into the aneurysm, through which occlusion agents are to be introduced into the aneurysm. In this way, the device prevents the escape of occlusive agents from the aneurysm. If a sufficiently large number of occlusion agents, usually coils, have been introduced into the aneurysm, they will interlock with each other and consequently impede each other from exiting the aneurysm, i.e., after complete filling of the aneurysm has taken place, further obstruction of the aneurysm neck may not be necessary. Such a technique is also called “jailing”. In the case of such an only temporarily inserted stent structure, a detachment point connecting to the insertion aid need not necessarily be arranged, and such a device is also considered to be included in the invention according to a further embodiment, provided that the stent structure at least partially carries the described coating.

Other types of flow diverters are so-called bifurcation flow diverters, which are positioned in front of aneurysms located at a vessel branching site (bifurcation). Such a bifurcation flow diverter or bifurcation implant is described, for example, in publication WO 2014/029835 A1. The distal section of such an implant is radially expanded relative to a more proximally situated section. Said distal section has a configuration that at least partially occludes the neck of the aneurysm. The described coating to prevent platelet adhesion and aggregation is also useful for such a bifurcation implant.

Also in the context of the indications of stenosis and aneurysm, the invention not only relates to the relevant device but also to a relevant method. During this procedure, the device is navigated to the target position, usually by means of a microcatheter. The device is released and assumes its expanded shape. This is brought about either by withdrawing the microcatheter in the proximal direction or by pushing the device forward out of the microcatheter in the distal direction. This is followed by detachment of the distal stent structure from the insertion aid such that the stent structure is released within the blood vessel and can remain there. The microcatheter can then be retracted in a proximal direction and removed from the blood vessel system. The accumulation of platelets and the formation of blood clots are effectively prevented when the stent structure intended for a stenosis treatment or the occlusion of an aneurysm remains in the blood vessel.

Irrespective of relevant embodiments, an essential feature of the invention is the biomimetic coating. As a rule, the device which serves as substrate is usually still covered by a carrier layer, which comprises adhesion promoters, through which the functional layer can be bonded to the substrate. Within the scope of the invention, preferred adhesion promoters are silane adhesion promoters. Alternatively, other adhesion promoters, for example polyolefinic adhesion promoters or adhesion promoters based on titanates or zirconates may also be employed.

Further examples of adhesion promoters include
Thiols and dithio compounds, particularly suitable for precious metal substrates
Amines and alcohols, especially suitable for platinum substrates
Carboxylic acids, especially suitable for silver substrates and aluminum substrates; the aluminum substrate may have an aluminum oxide surface
Phosphonic acids (phosphonates), especially suitable for iron, iron oxide, titanium and titanium dioxide substrates
Complexing adhesion promoters, particularly chelates, which to some extent also bind non-covalently to substrates, particularly suited for various metal and metal oxide substrates

The adhesion promoters should comprise functional groups via which the adhesion promoter is capable of reacting with the functional layer, so that as a rule covalent bonding is possible. Depending on the relevant device material, bonding of the adhesion promoter with the device can also be covalent.

For example, an appropriate adhesion promotion may be achieved by silanization, that is the chemical bonding of silicon compounds, in particular silane compounds, to at least parts of their surface. On surfaces, silicon and silane compounds attach, for example, to hydroxy and carboxy groups.

Preferably, the substrate shall allow bonding to take place with an adhesion promoter. For the purposes of this application, such substrates shall be referred to as “coatable substrate”. Accordingly, coatable substrates comprise substrates whose surface is sufficiently reactive and/or sufficiently activatable to form bonds at least partially with an adhesion promoter or even directly with the functional layer.

Within the meaning of the present invention, coatable substrates may therefore be of very different nature and, in particular, comprise oxidizable substrates and combinations thereof. This includes, for example, metals such as nickel, titanium, platinum, iridium, gold, cobalt, chromium, aluminum, iron or alloys, as well as combinations thereof. For example, a metal can also be coated with another metal, in which case the coating claimed by the invention is in turn applied to the outer metal layer, preferably comprising the carrier layer and the functional layer. Substrates in which the basic metal is covered by an oxide layer shall also count among coatable metals. Other coatable substrates are glasses.

A particularly preferred embodiment relates to a device that is provided in whole or in part with a gold coating through which X-ray visibility is ensured. In particular, this enables the dilation of the device in the blood vessel to be visualized, so that the treating physician can recognize whether the expansion is taking place as desired. This is of special advantage in the event an implant is intended for the treatment of vasospasm. The coating proposed by the invention is then applied to the gold coating, comprising the functional layer and in most cases also a carrier layer. The base material of the device to which the gold coating is applied can be a customary metal or customary metal alloy intended for relevant medical devices, such as a nickel-titanium alloy, a cobalt-chromium alloy, or stainless steel.

Coatable substrates within the meaning of the present invention may also be various plastics, such as polyamides (PA), polytetrafluoroethylene (PTFE), expanded polytetrafluoroethylene (ePTFE), polylactides (PLA), polyester, polyether, polyurethane, polyolefins, as well as relevant block copolymers. In the field of medical engineering, those skilled in the art are familiar with a large number of suitable plastics. Whereas an adhesion promoter is usually needed for metallic or oxidic surfaces, it is not always needed for polymers used as substrates.

For example, suitable adhesion promotion may be achieved by silanization, i.e., via a chemical bonding of silicon compounds, in particular silane compounds, to at least parts of their surface. On surfaces, silicon and silane compounds bind to hydroxy and carboxy groups, for example.

Polyolefins can also be used as adhesion promoters, including chlorinated polyolefins (CPO) or acrylated polyolefins (APO).

Silane compounds within the meaning of the invention are all those compounds which follow the general formula RₘSiXₙ (m, n=0-4, where R stands for organic radicals, in particular alkyl, alkenyl or aryl groups, and X stands for hydrolyzable groups, in particular OR, OH or halogen, with R=alkyl, alkenyl or aryl). In particular, the silane may have the general formula RSiX₃. Moreover, for the purposes of the invention relevant compounds with several silicon atoms also count among silane compounds. In particular, silane derivatives in the form of organosilicon compounds are regarded as silane compounds in accordance with the invention. Accordingly, silane compounds within the meaning of the invention are not only substances having a silicon backbone and hydrogen and being designated as silanes.

Preferably, the matrix of the functional layer is covalently bonded to the carrier layer or substrate and is preferably synthesized by graft polymerization, with the functional layer being produced on the carrier layer or substrate. The polymerization of the applied monosaccharides, with reduction and oxidation products of monosaccharides also being understood as such, in particular sugar alcohols (alditols), essentially occurs preferably only on the carrier layer/the substrate or, resp., within the functional layer. Sugar alcohols (alditols) are reduction products of sugars in which an aldehyde function has been reduced to an alcohol.

With respect to the purposes of the invention, it is irrelevant in which form (graft) polymerization occurs. Therefore, growth of the side chains may in particular start from a main chain. This approach is also referred to as “grafting from”. Similarly, it is possible that the side chains have already started oligomerization or polymerization and the already growing side chains link to the main chain (“grafting onto”). Furthermore, main and side chains that have already been oligomerized or polymerized can also congregate (“grafting through”).

Preferably, the functional layer substantially comprises a complex, highly branched, hydrophilic matrix comprising a plurality of molecules each having a main chain as polymer backbone with a plurality of side chains each. The main and/or side chains may form bonds with other main and/or side chains. Other matrix-forming mono-, oligo- and polymers can be integrated into these main and side chains without being themselves covalently bonded to the carrier layer.

The main chain may comprise at least partially polymerized vinyl, allyl, acrylic or methacrylic compounds or derivatives thereof and/or isomers thereof or combinations thereof.

The side chains particularly comprise mono- and/or oligosaccharides, with reduction products of mono- or oligosaccharides also being understood as such, in particular sugar alcohols (alditols). In addition, oxidized mono- and/or oligosaccharides may also occur, with the oxidized form also being understood as mono- or oligosaccharides for the purposes of the invention.

The device proposed by the invention comprises at least a coated substrate, said coating preferably comprising a carrier layer existing on the substrate and a functional layer located on the carrier layer. The carrier layer essentially comprises the adhesion promoters, which in most cases are covalently bonded to the substrate. Moreover, non-covalently bonding adhesion promoters are also known, for example those that attach to the substrate via a complex bond. Preferred adhesion promoters are silicon compounds and polyolefinic adhesion promoters. According to a preferred embodiment, the functional layer comprises at least one functionalized sugar alcohol, via which the functional layer is covalently bonded to the carrier layer.

A preferred sugar alcohol of the functional layer corresponds in its non-functionalized form to a sugar alcohol with the molecular formula C₆H₁₄O₆, for example sorbitol, and/or its derivatives, for example sorbitan. Other sugar alcohols can be mannitol, lactitol, xylitol, threit, erythritol or arabitol. The structure of sorbitol is shown below:

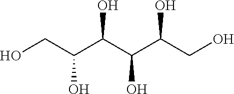 [SMILES: OC[C@@H](O)[C@@H](O)[C@H](O)[C@@H](O)CO]

“In its non-functionalized form” means that the molecular formula referred to represents the molecular formula of the non-functionalized sugar alcohol, but should also include, where appropriate, its derivatives and/or isomers thereof. Functionalization shall be understood to indicate the introduction of a function into the compound permitting an attachment to the substrate, the carrier layer and/or to compounds already attached previously to the carrier layer or substrate.

The functional layer according to the invention comprises functionalized variants of the sugar alcohol of molecular formula C₆H₁₄O₆ and/or its derivatives and/or its isomers. The functional layer can in particular comprise a complex matrix that can be created by polymerization of the applied, functionalized sugar alcohols.

In addition to the definition of the term generally used in chemistry as “derived substance of similar structure”, and for the purposes of the invention, derivatives are to be understood as all cyclic and heterocyclic compounds derivable from the substance by dehydration. An example here is sorbitan or sorbitan anhydride, which is formed by splitting off a water molecule from sorbitol. It thus represents the anhydride of sorbitol. Another example is isosorbide, which is obtained by splitting off an additional water molecule.

Preferably, the sugar alcohol or the sugar alcohols are functionalized via at least one reactive group, wherein the reactive group preferably comprises a reactive multiple bond, in particular a double bond, and wherein this reactive double bond is preferably an acrylic group. Other functional groups suitable for polymerization, which must not necessarily have a reactive double bond, are known to the person skilled in the art and include, for example, methacrylic groups, vinyl groups or allyl groups.

Preferably, the sugar alcohols of the functional layer are at least partially polymerized with each other.

The device preferably comprises at least one substrate provided with a coating, with said coating comprising a functional layer. The functional layer comprises at least one functionalized monosaccharide, with the monosaccharides being covalently attachable to the carrier layer and oligomerizing or polymerizing taking place only upon bonding to the carrier layer. It has been found that in this way a functional layer is created that is particularly similar to the natural glycocalyx. The structure of the inventive coating, in which oligomerization or polymerization of the saccharides takes place only upon bonding to the substrate, differs significantly from a coating in which ready-made polymers are applied to a surface. In particular, the coating created also differs from prior art coatings in that it has an especially low layer thickness, which usually amounts to 100 nm. In most cases, the thickness of the coating ranges between 10 and 100 nm. This is also associated with the advantage that the mechanical properties of the coated medical devices are only marginally affected if at all, i.e., the elasticity, the ability to expand after release, to exert a radial force on the blood vessel, etc. are all retained.

Preferably, the coating comprises a carrier layer located on the substrate, with the functional layer in turn being bonded to the carrier layer. The bonds formed may be covalent bonds in particular, however, may also be other bonds such as complex bonds. The carrier layer essentially comprises the adhesion promoters bonded to the substrate. Preferred adhesion promoters are silicon compounds and polyolefinic adhesion promoters.

The monosaccharide of the functional layer preferably comprises at least one sugar alcohol and/or its derivatives and/or its isomers.

The solution from which the functional layer of the coating proposed by the invention is made up may thus comprise individual or a multitude of the following substances:
(1) Sorbitol acrylates (composed of one or several acrylate group(s)), the acrylate group(s) of which may be located at different positions.

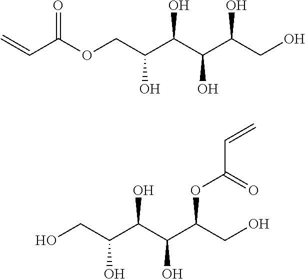 [SMILES: C=CC(=O)OC[C@@H](O)[C@@H](O)[C@H](O)[C@@H](O)CO ; C=CC(=O)O[C@@H](CO)[C@@H](O)[C@H](O)[C@H](O)CO]

Sorbitol Monoacrylates

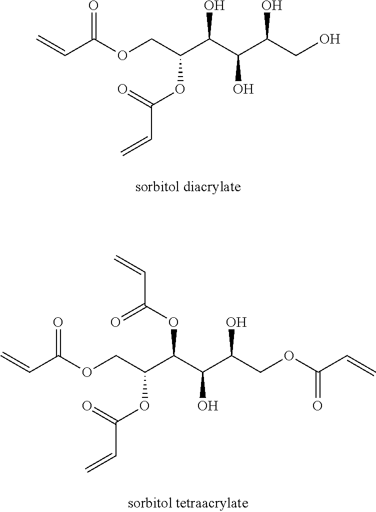 [SMILES: C=CC(=O)OC[C@@H](OC(=O)C=C)[C@@H](O)[C@H](O)[C@@H](O)CO ; C=CC(=O)OC[C@H](O)[C@@H](O)[C@H](OC(=O)C=C)[C@@H](COC(=O)C=C)OC(=O)C=C]

(2) Sorbitol acrylates (comprising one or a multitude of acrylate group(s)), wherein the sorbitol acrylates may be partially oxidized and may comprise an aldehyde, keto and/or carboxy group.

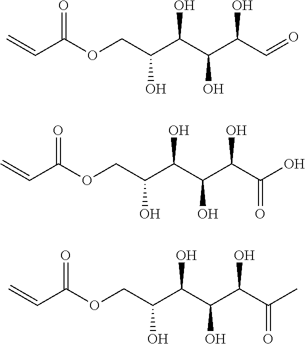 [SMILES: C=CC(=O)OC[C@@H](O)[C@@H](O)[C@H](O)[C@@H](O)C=O ; C=CC(=O)OC[C@@H](O)[C@@H](O)[C@H](O)[C@@H](O)C(=O)O ; C=CC(=O)OC[C@@H](O)[C@@H](O)[C@H](O)[C@@H](O)C(C)=O]

(3) Sorbitol acrylates (with one or a multitude of acrylate group(s)), which may comprise further reactive groups such as carboxy groups.

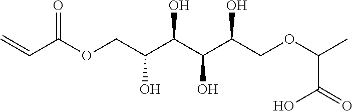 [SMILES: C=CC(=O)OC[C@@H](O)[C@@H](O)[C@H](O)[C@@H](O)COC(C)C(=O)O]

(4) Anhydrides, for example sorbitan (mono) acrylate comprising a polymerizable group.

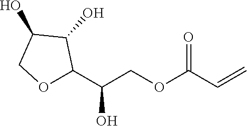

(5) Sorbitol having a non-polymerizable group, for example a carboxy group.

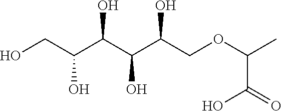 [SMILES: CC(OC[C@H](O)[C@@H](O)[C@H](O)[C@H](O)CO)C(=O)O]

(6) Complex sorbitol compounds that are not polymerizable but can be incorporated into the polymer matrix of the functional layer.

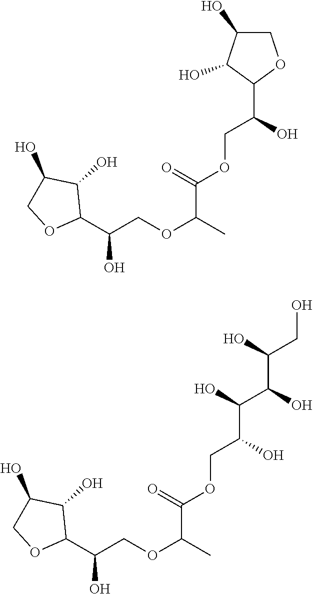 [SMILES: CC(OC[C@@H](O)C1OC[C@@H](O)[C@@H]1O)C(=O)OC[C@H](O)C1OC[C@H](O)[C@H]1O ; CC(OC[C@@H](O)C1OC[C@@H](O)[C@@H]1O)C(=O)OC[C@@H](O)[C@@H](O)[C@H](O)[C@@H](O)CO]

The structure of the functional layer can be varied via the specific composition of the substances. For example, it is possible to produce more tightly meshed functional layers by increasing the proportion of crosslinkers, or more lightly crosslinked functional layers with longer linear regions by using a lower proportion of crosslinkers.

Another advantage of the coating according to the invention is that, via the intermediate step of adhesion promotion, the coating covers only those surfaces and structures of the devices that can be activated for the relevant adhesion promoters and, in particular, have also been activated. When applying the functional coating solution, it is thus possible to immerse the complete device in the functional coating solution without having to additionally protect areas that are not to be coated.

Such a selective coating, respectively a selectively implemented coating method, offers advantages described hereinbefore for a plurality of devices and at least for those devices which are made of different materials, in which case the relevant coating is to be applied only on some of these materials.

The coating proposed by the present invention enables the coating method to activate only those parts/areas of the device that are intended to subsequently carry the functional layer. It is also conceivable that the device is already designed in such a way that the parts to be coated comprise substances that are capable of being activated for adhesion promotion.

Devices provided with the coating claimed by the invention are particularly suitable for endovascular, neurovascular and cardiovascular fields of application; however, the inventive coating for a device can always be expediently applied on all devices that come into contact with blood.

Any and all statements made with respect to the devices shall equally apply in the same way as well to the relevant methods and vice versa.

### TESTS

The inventive coating was subjected to a series of in vitro tests in order to ascertain the effectiveness of the coating proposed by the present invention. For this purpose, one uncoated small nitinol plate specimen and one small nitinol plate specimen silanized according to the invention and subsequently coated with polymerized sorbitol acrylate were incubated with heparinized whole blood for a period of 10 minutes in each test series. The adhesion of platelets was then determined by fluorescence microscopy using fluorescence-labelled CD61 antibodies.

The adhesion of platelets/thrombocytes to the nitinol plate specimens coated in accordance with the invention was found to be significantly lower than that of the uncoated nitinol plates.

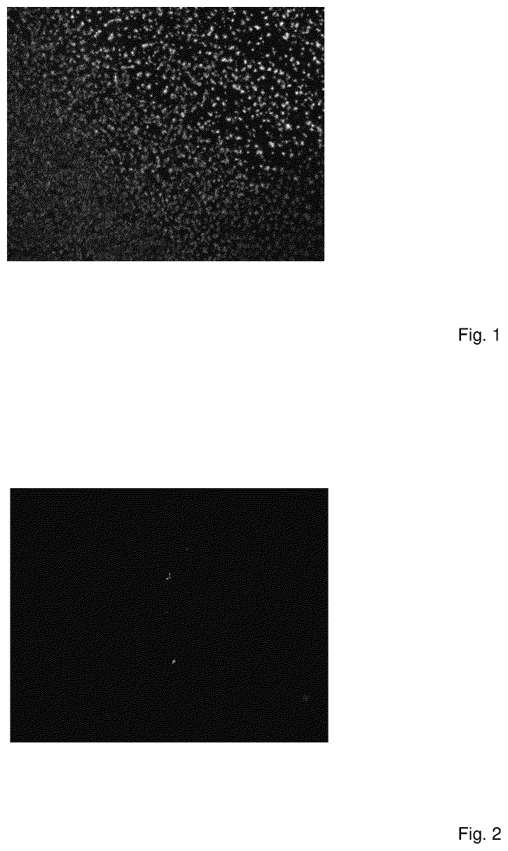

FIG. 1 shows an uncoated small nitinol plate specimen after 10 minutes incubation time with heparinized whole blood at 10× magnification under the fluorescence microscope. The adhesion of a multitude of CD61 positive platelets is clearly visible.

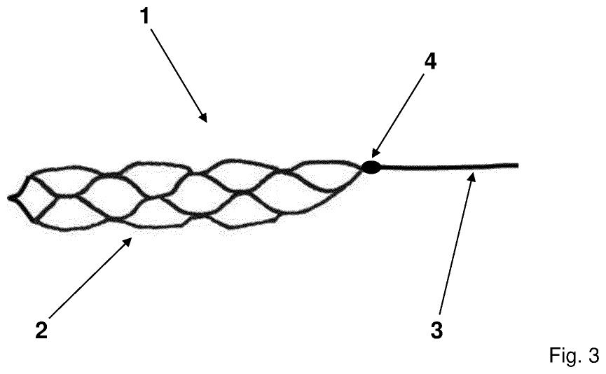

FIG. 2 shows a coated small nitinol plate specimen after 10 minutes incubation time with heparinized whole blood at 10× magnification under the fluorescence microscope. Only a few attached CD61 positive platelets can be recognized.

FIG. 3 shows an exemplary side view of the device 1 according to the invention. The device has a stent structure 2 and an insertion aid 3 in the form of an insertion/pusher wire. In this example, stent structure 2 is fabricated by laser cutting and comprises struts that in their entirety form a continuous honeycomb structure. The insertion aid 3 is eccentrically connected, i.e., in the peripheral region, to the stent structure 2 at its proximal end via a detachment point 4. By applying an electrical voltage to the detachment point 4, the stent structure 2 can be disconnected from the insertion aid 3 and permanently implanted in the blood vessel.

1. A device with a stent structure which is intended for insertion into blood vessels of the human or animal body, wherein the stent structure assumes an expanded state in which it is in contact with the inner wall of the blood vessel and has a diameter-reduced state in which it is movable through the blood vessel within a microcatheter, wherein the stent structure, preferably at its proximal end, is connected to an insertion aid, and wherein the device is deployable for the treatment of a vasospasm wherein:
the stent structure is designed so as to be detachable from the insertion aid, wherein at least portions of the stent structure are provided with a coating and
said coating comprises a functional layer, with said functional layer comprising at least one sugar alcohol and/or being formed by an oligo- or polymerization of monosaccharides functionalized with polymerizable groups.

2. A device according to claim 1, wherein the stent structure is composed of interconnected struts or wires forming a mesh structure.

3. A device according to claim 1, wherein the stent structure is self-expanding and autonomously changes to the expanded state after release from the microcatheter.

4. A device according to claim 2, wherein the struts or wires in the event of an essentially rectangular cross section have a height and width of between 30 and 300 μm and in the event of a circular cross section have a diameter ranging between 30 and 300 μm.

5. A device according to claim 2, wherein no struts or wires are arranged at the proximal or distal end or both at the proximal and distal end in the center of the stent structure.

6. A device according to claim 1, wherein the force exerted radially outward by the expanded stent structure ranges between 2 and 30 N/m, preferably between 5 and 10 N/m, based on a diameter of the stent structure of 2.00 mm.

7. A device according to claim 1, wherein the stent structure has a proximal, a middle and a distal portion, wherein the proximal portion comprises the proximal end at which the stent structure is connected to the insertion aid, and wherein the expanded stent structure outside the proximal end exerts a substantially constant radial force along its entire length.

8. A device according to claim 7, wherein the struts or wires have a larger cross section in the proximal and distal section than in the middle section.

9. A device according to claim 7, wherein the density of the struts or wires is higher in the proximal and distal section than in the middle section.

10. A device according to claim 1, wherein the monosaccharide of the functional layer is functionalized in a form not bonded to the device via at least one reactive multiple bond.

11. A device according to claim 10, wherein the reactive double bond is a constituent of a (meth)acrylic group.

12. A device according to claim 1, wherein the stent structure is provided with a gold coating under the functional layer.

13. A device according to claim 1, wherein the coating comprises a carrier layer located on the stent structure with an adhesion promoter, and the functional layer is bonded to the carrier layer.

14. A device according to claim 13, wherein the adhesion promoter is a silicon compound, in particular a silane compound, or a polyolefin.

15. A method for the treatment of a vasospasm, wherein the stent structure of a device (1) according to claim 1 is brought to the position of the vasospasm with the aid of the insertion aid and expanded, and a detachment of the stent structure is carried out.

16. A device according to claim 1, wherein the stent structure at its proximal end is connected to the insertion aid.

17. A device according to claim 6, wherein the force exerted radially outward by the expanded stent structure ranges between 5 and 10 N/m, based on a diameter of the stent structure of 2.00 mm.

18. A device according to claim 10, wherein the reactive multiple bond is a double bond.

19. A device according to claim 14, wherein the silicon compound is a silane compound.
